(19) United States
(12) Patent Application Publication
(10) Pub. No.: US 2025/0152619 A1
(43) Pub. Date: May 15, 2025

(54) POLYIMIDAZOLIUM-BASED CATIONIC ANTIMICROBIAL POLYMERS FOR NOVEL MASTITIS PROPHYLACTIC SOLUTIONS

(71) Applicants: Nanyang Technological University, Singapore (SG); Massachusetts Institute of Technology, Cambridge, MA (US)

(72) Inventors: Bee Eng Mary CHAN, Singapore (SG); Paula HAMMOND CUNNINGHAM, Cambridge, MA (US); Kaixi ZHANG, Singapore (SG); Michelle Elizabeth FERGUSON, Singapore (SG)

(21) Appl. No.: 18/727,019

(22) PCT Filed: Jan. 4, 2023
(86) PCT No.: PCT/SG2023/050007

§ 371 Date: Jul. 5, 2024

Related U.S. Application Data
(60) Provisional application No. 63/266,477, filed on Jan. 6, 2022.

(51) Int. Cl. A61K 31/787; A61K 47/10; A61P 31/04; C08G 73/06

(52) U.S. Cl. CPC A61K 31/787; A61K 47/10; A61P 31/04; C08G 73/0616

(57) ABSTRACT

The present invention relates to a composition comprising a polyimidazolium (PIM) compound for use in the treatment and/or prevention of mastistis in a mammal, and to methods of use.

### FIELD OF THE INVENTION

The present invention relates to a composition comprising a polyimidazolium (PIM) compound for use in the prophylaxis or treatment of mastistis in mammals, and methods of using same.

### BACKGROUND OF THE INVENTION

Mastitis can affect essentially all lactating mammals, but is especially problematic for dairy cattle, sheep and goats. Mastitis is an inflammation of the mammary gland (udder). It can be caused by physical injury or stress or by bacteria which invade the mammary gland. The bacteria which are known to cause mastitis in cows, sheep and goats are Streptococcus sp., Staphylococcus sp., Pasteurella sp., and coliforms, such as E. coli.

Mastitis in dairy cows is caused by bacteria introduced either during the milking process or through environmental contact. Examples of this are contamination from milking machines, milking staff, and liquid manure contamination from dirty stalls. Mastitis is one of the most common diseases affecting dairy herds around the world and is viewed as a costly dairy production disease with variable levels of economic loss². Economic losses associated with mastitis result primarily from a decrease in milk production, discarded milk, veterinary services, veterinary treatment costs, drug costs, decreased cow sales value, culling of continuously infected cows, labour, and penalties for milk quality. The Indian dairy industry suffers an annual loss of approximately USD 1200 million due to mastitis, while that loss for the United States is nearly USD 1800 million.

The prevention and control of mastitis in cows requires consistent disinfection of the cowshed facilities, a proper milking process, and the separation of infected animals. A wide range of antibiotics such as lincomycin (Lincocin® Forte S), amoxicillin (Nisamox®) novobiocin (Tetra-Delta™) and ampicillin (Kloxerate Plus) have been used to treat mastitis in affected mammals over the past 40 years. Due to the emergence of antibiotic resistance and the alarming rate of its dissemination, non-antibiotic measures are in urgent need to contain mastitis, particularly in dairy cows. Most of the preventative products in use are broad-spectrum disinfectants, with the common active ingredients being chlorohexidine, Polyvinylpyrrolidone (PVP) iodine (also known as povidone-iodine) and sodium hypochlorite (Table 1).

TABLE 1
Commercially available mastitis-preventing products

| Product Name | Company | Active ingredient | Dosing regime | Disadvantage |
|---|---|---|---|---|
| TeatX | Deosan | Chlorhexidine | Daily post | Toxic |
| | | | milking | |
| TriodeX | Deosan | PVP iodine | Daily post | Toxic |
| | | | milking | |
| Zoom Hypo | Deosan | sodium | Daily before | Toxic and irritating |
| Chlor | | hypochlorite | milking | |
| Iodine Teat | Stearns | 0.5% Iodine | Daily post | Less effective; iodine |
| Dip | | | milking | residual in milk |
| GLADIATOR | BouMatic | 1% chlorine | Daily post | Toxic and irritating |
| BARRIER | | dioxide | milking | |
| BLUEMAX | BouMatic | biphenyl-2-ol | Daily post | Toxic, irritating and |
| BARRIER | | | milking | potentially carcinogenic |
| Eco-Flex | ECOLAB | Glycerin, sorbitol, | Daily post | Less effective; iodine |
| Non-Iodine | | lanolin, and | milking | residual in milk |
| Teat Dip | | propylene glycol | | |
| REMAIN | ECOLAB | Bronopol-based | Daily post | Less effective (as MIC is |
| GOLD non- | | | milking | higher) |
| iodine dip | | | | |
| Wash & Prep | ECOLAB | lactic acid and | Daily pre | Irritating; used only for |
| RTU | | linear alkylate | milking | skin cleaning before |
| | | sulfonates | | milking |

However, most of the preventative products suffer from potential problems such as low efficacy in the presence of milk, toxicity and irritation to teats, potential carcinogenicity, and/or residual in milk.

There is a need for improved mastitis-prevention antimicrobial products.

### SUMMARY OF THE INVENTION

In a previous study [Zhong, W. et al., Proceedings of the National Academy of Sciences, 117(9):31376-31385 (2020)], a novel series of cationic antimicrobial polymers which are main chain alkylated polyimidazoliums (PIMs) were reported to exhibit broad-spectrum activity against bacteria and low acute cytotoxicity against mammalian cells. In the present invention, two specific main chain alkylated PIMs were evaluated, specifically PIM1 and PIM1D, for their efficacy in preventing mastitis. PIM1 and PIM1D achieved more than 5 log₁₀ reduction of mastitis-causing pathogens within 10 minutes. Both compounds showed superior bactericidal activity compared with the commercially used antiseptic chlorhexidine, with much better biocompatibility against mammary cells in vitro. Furthermore, the in vivo efficacy of PIM1 and PIM1D was confirmed in a farm trial as post-milking teat dip products to prevent mastitis of milking cows upon 5-day continuous contagious challenge with Staphylococcus aureus ATCC 49525. In summary, this novel antimicrobial PIM series retains excellent efficacy in the presence of complex milk solutions that could fail many other cationic antimicrobials.

In a first aspect, the present invention provides a method of prophylaxis or treatment of mastitis in a mammal, comprising administering an efficacious amount of a composition comprising one or more polyimidazolium (PIM) compounds to said mammal, wherein the one or more polyimidazolium (PIM) compounds is selected from the group consisting of:

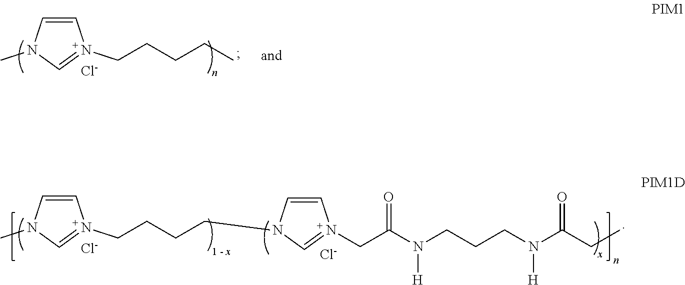

In some embodiments, for PIM1 n is a number selected from 2 to 10 and the number average molecular weight is from 1,000 to 2,000 Daltons; and for PIM1D n is a number selected from 2 to 10, x is about 25%, and the number average molecular weight is from 1,000 to 2,000 Daltons.

In some embodiments, for PIM1 n=9 and the number average molecular weight is 1562 Daltons; and for PIM1D n=10, x is about 25% and the number average molecular weight is 1856 Daltons.

In some embodiments, the polyimidazolium compound is a salt, such as a chloride salt.

The amount of the polyimidazolium compound of the invention in any pharmaceutical formulation used in accordance with the present invention should be sufficient to effect a therapeutic response in the mammal over a reasonable timeframe. One skilled in the art will recognize that the selection of the exact dose and composition and the most appropriate delivery regimen will depend on various factors, such as the severity of the condition to be treated, the particular subject or species of mammal to be treated, as well as the compound(s) which is/are employed. In any event, the amount of polymer or copolymer of the invention in the formulation may be determined routinely by the skilled person.

In some embodiments, the composition comprises at least 0.05% w/v polyimidazolium compound.

In some embodiments, the composition further comprises glycerol.

In some embodiments, the composition comprises glycerol in an amount of 10% w/v or less.

In some embodiments, the mammal is selected from the group comprising cows, pigs, goats, sheep and humans.

In some embodiments, the mammal is a cow.

In some embodiments, the composition is applied to one or more teats of said mammal.

In some embodiments, the composition is applied daily post-milking.

In a second aspect, the present invention provides a composition comprising one or more polyimidazolium (PIM) compounds for use in a method for prophylaxis or treatment of mastitis in a mammal, wherein said one or more polyimidazolium (PIM) compounds are selected from the group consisting of

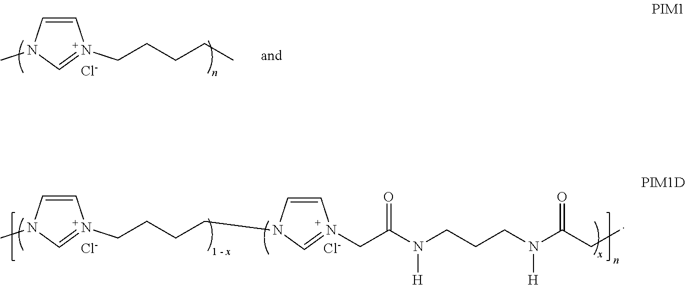

In some embodiments, for PIM1 n is a number selected from 2 to 10 and the number average molecular weight is from 1,000 to 2,000 Daltons; and for PIM1D n is a number selected from 2 to 10, x is about 25%, and the number average molecular weight is from 1,000 to 2,000 Daltons.

In some embodiments, for PIM1 n=9 and the number average molecular weight is 1562 Daltons; and for PIM1D n=10, x is about 25% and the number average molecular weight is 1856 Daltons.

In some embodiments, the polyimidazolium compound is a salt, such as a chloride salt.

In some embodiments, the polyimidazolium salt is a chloride salt.

In some embodiments, the composition comprises at least 0.05% w/v polyimidazolium compound.

In some embodiments, the composition further comprises glycerol.

In some embodiments, the composition comprises glycerol in an amount of 10% w/v or less.

In some embodiments, the mammal is selected from the group comprising cows, pigs, goats, sheep and humans.

In some embodiments, the mammal is a cow.

In a third aspect, the present invention provides a use of a polyimidazolium (PIM) composition of the second aspect in the manufacture of a medicament for the prophylaxis or treatment of mastitis in a mammal.

In some embodiments, the mammal is selected from the group comprising cows, pigs, goats, sheep and humans.

In some embodiments, the mammal is a cow.

### BRIEF DESCRIPTION OF FIGURES

FIG. 1 shows Log reduction of 0.5%, 0.1%, 0.05% and 0.01% w/v PIM1 and 0.5%, 0.1%, 0.05% and 0.01% w/v chlorhexidine in 10 minutes in the presence of 18% milk against (A) S. aureus (B) S. uberis (C) E. coli.

FIG. 2 shows a log reduction of 0.5%, 0.1%, 0.05% and 0.01% w/v PIM1 and 0.5%, 0.1%, 0.05% and 0.01% w/v chlorhexidine in 5 min (A) or 30 min (B) against S. aureus.

FIG. 3 shows a log reduction of 0.5%, 0.1%, 0.05% and 0.01% w/v PIM1 and 0.5%, 0.1%, 0.05% and 0.01% w/v chlorhexidine in 5 min (A) or 30 min (B) against S. uberis.

FIG. 4 shows a log reduction of 0.5%, 0.1%, 0.05% and 0.01% w/v PIM1 and 0.5%, 0.1%, 0.05% and 0.01% w/v chlorhexidine in 5 min (A) or 30 min (B) against E. coli.

FIG. 5 shows in vitro cytotoxicity study of PIMs and commercial antiseptic chlorhexidine. Human mammary MCF-7 cells exposed to a concentration range of 0.001%-0.1% w/v PIM1 (solid black box) and PIM1D (solid grey box) showed much reduced toxicity compared to 0.001%-0.1% w/v chlorhexidine (diamond).

FIG. 6 shows the timeline and experimental setup of the farm trial, including 10 days acclimation, followed by a 5 day safety trial of the PIM1 and PIM1D based teat dips, and then a 7 day mastitis pathogen challenge trial.

FIG. 7 shows teat images after a 5-day safety trial of continuous application of PIM1 (A) and PIM1D (B) based teat dip.

FIG. 8 shows milk composition changes, including protein (A), fat (B), solid non-fat (SNF) (C) and Somatic cell counts (SCCs) (D) content, before and after 5-day consecutive teat dip application of PIM1 (solid black box) or PIM1D (solid grey triangle).

FIG. 9 shows the Delvo Test results of (A) teat surface and (B) milk samples.

FIG. 10 shows bacteria count in milk over time upon exposure to repeated S. aureus for (A) 0, (B) 3 and (C) 5 days with glycerol (solid circles), PIM1 (solid squares) or PIM1D (solid triangles) treatment. Each dot indicates one milk sample from one teat (also known as one quarter). The criteria for mastitis is defined as >500 CFU/mL in milk samples (indicated by the dotted line).

FIG. 11 shows SCC (somatic cell counts) in milk samples upon bacteria challenge for 0, 3 and 5 days with glycerol (solid circles), PIM1 (solid squares) or PIM1D (solid triangles) treatment. Each data point indicates the sample from one cow.

FIG. 12 shows that milk compositions change upon bacteria challenge. (A) protein, (B) fat and (C) SNF.

### DETAILED DESCRIPTION OF THE INVENTION

Bibliographic references mentioned in the present specification are for convenience listed in the form of a list of references and added at the end of the Examples. The whole content of such bibliographic references is herein incorporated by reference.

### Definitions

Unless defined otherwise, all technical and scientific terms used herein have the same meaning as is commonly understood by one of skill in the art to which the invention belongs. Certain terms employed in the specification, examples and appended claims are collected here for convenience.

It must be noted that as used herein and in the appended claims, the singular forms “a,” “an,” and “the” include plural reference unless the context clearly dictates otherwise. Thus, for example, a reference to “a target sequence” includes a plurality of such target sequences, and a reference to “an enzyme” is a reference to one or more enzymes and equivalents thereof known to those skilled in the art, and so forth.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

References herein (in any aspect or embodiment of the invention) to polyimidazolium compounds includes references to such compounds per se, to tautomers of such compounds, as well as to salts or solvates of such compounds.

Salts that may be mentioned include acid addition salts and base addition salts. Such salts may be formed by conventional means, for example by reaction of a free acid or a free base form of a polyimidazolium compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of salts include acid addition salts derived from mineral acids and organic acids, and salts derived from metals such as sodium, magnesium, or preferably, potassium and calcium. Advantageously, the polyimidazolium compound of the invention are chloride salts.

The term “treatment”, as used in the context of the invention refers to prophylactic, ameliorating, therapeutic or curative treatment.

### EXAMPLES

Standard molecular biology techniques known in the art and not specifically described were generally followed as described in Green and Sambrook, Molecular Cloning: A Laboratory Manual, Cold Spring Harbour Laboratory, New York (2012).

### Example 1

### Methods

### Synthesis of Main Chain Alkylated Polyimidazoliums (PIMs)

The PIMs useful for the invention can be synthesized according to the methods described in Zhong, W. et al., [Proceedings of the National Academy of Sciences, 117(9): 31376-31385 (2020)].

Briefly, an aqueous acidic solution of diamine (100-mmol total) was maintained in an ice water bath for 30 min, after which we added a mixture of formaldehyde (8.12 g, 100 mmol) and glyoxal (14.51 g, 100 mmol) dropwise. The reaction mixture was refluxed for 4.5 h at 80° C. with the exception of PIM5 which was refluxed for 12 h at 120° C. During reflux the solutions changed from colorless to yellowish. We removed most of the solvent and the unreacted monomers by rotary evaporation to give a yellow viscous oil, which was diluted with water and dialyzed against acidified water, pH 3 to 4 (1-kDa-cutoff Spectra/Por6 dialysis membrane, Repligen) for 1 d. The diamine for synthesis of PIM0 was 1,3-diaminopropane; for PIM1, 1,4-diaminobutane; PIM2, 1,6-diaminohexane; PM3, 1,8-diaminooctane; PIM4, 1,5-diamino-2-methylpentane; PIM5, 2,2′-(ethylenedioxy)bis(ethylamine); PIM6, 4,7,10-trioxa-1,13-tridecanediamine; and PIM7, L-lysine.

Synthesis of PIM1D was more difficult than synthesis of PIM0-7. A mixture of 2 diamines, Diamine A (N, N′-(propane-1,3-diyl)bis(2-aminoacetamide) and Diamine B (1,4-diaminobutane), which are degradable and non-degradable respectively, were used for the PIM1D synthesis. Diamine B was purchased from Merck & Co. (USA). Diamine A was synthesized as described below:

### Diamine A Synthesis.

N-(3-dimethylaminopropyl)-N′-ethylcarbodiimide hydrochloride (EDC·HCl) (14.58 g, 76.1 mmol) and 1-Hydroxybenzotriazole (HOBt) (10.70 g, 79.14 mmol) were added to a solution of N-(tert-butoxycarbonyl) glycine (Boc-Gly-OH) (8.0 g, 45.66 mmol) in anhydrous dimethylformamide (DMF) (25 ml), at 0° C. (ice water) with stirring (30 min). 1,3-diaminopropane (1.28 ml, 15.22 mmol) kept at room temperature was added dropwise to the solution at 0° C. over 10 min after which, the mixture was moved to room temperature and continuously stirred for 48 h. 50 ml water was then added and the product was extracted three times with 50 ml of diethyl ether (Et₂O) for each extraction. The pooled extracts were washed with water (3 times 50 ml) and then washed once with saturated sodium chloride solution (50 ml). The diethyl ether layer was dried with anhydrous sodium sulfate (Na₂SO₄) (about 50 g). The sodium sulfate was then removed by filtration, and the filtrate was concentrated by rotary evaporation (20 min at 50° C., 120 rpm). The residue was dried under vacuum at room temperature for overnight. The dried residue was dissolved in anhydrous dichloromethane (CH₂Cl₂) (30 ml) and then kept at 0° C. and 8 ml of trifluoroacetic acid (TFA) was added dropwise over 10 min, after which the reaction mixture was stirred at room temperature for 12 h. The reaction products were concentrated by rotary evaporation at 50° C. for 10 min at 120 rpm. 50 ml of toluene was added and the solution was subjected to further rotary evaporation at 50° C. for 30 min at 120 rpm. The residue was purified by silica gel 60 column chromatography with successive eluents of (i) 30% methanol (MeOH) in CH₂Cl₂ (500 ml) to remove impurities followed by (ii) 2% TFA in MeOH (1000 ml), to yield the degradable diammonium TFA salt A (3.0 g, 7.20 mmol). The obtained intermediate compound in PIM1D synthesis was characterized by NMR: 1H NMR (300 MHz, DMSO-d6, 25° C. [ppm]): δ 8.55 (t, J=5.4 Hz, 2H), 8.18 (br s, 6H), 3.53 (s, 4H), 3.14 (q, J=6.3 Hz, 4H), 1.54-1.63 (m, 2H). 13C NMR (75 MHz, DMSO-d6, 25° C. [ppm]): δ 166.14, 159.58 (—CO—CF₃), 159.16 (—CO—CF₃), 158.74 (—CO—CF₃), 158.32 (—CO—CF₃), 123.24 (—CF₃), 119.29 (—CF₃), 115.33 (—CF₃), 111.38 (—CF₃), 40.26 (—CO—CH₂—), 36.73 (—NH—CH₂—), 28.86 (—CH₂—CH₂—).

To obtain the Diamine A, triethylamine (Et₃N) (1 ml) was added to a stirred solution of diammonium TFA salt A (400 mg, 0.96 mmol) in MeOH (4 ml) maintained at 0° C. After stirring the reaction mixture for 30 min at room temperature, volatiles were evaporated under rotary evaporator (50° C., 20 mins, 120 rpm) and the residue was dried under vacuum at room temperature for 20 min to yield Diamine A, which was immediately used for following polymerization step.

### PIM1D Synthesis

PIM1D synthesis was carried out using 4:6 molar ratios of Diamine A to Diamine B (1,4-diaminobutane). A mixture of glyoxal 40 wt % (349 mg, 2.4 mmol) and formaldehyde 37 wt % (195 mg, 2.4 mmol) in glacial acetic acid (AcOH) and tetrahydrofuran (THF) (3:1.25 ml) at 0° C. (ice water) was prepared. A second solution of Diamine A (181 mg, 0.96 mmol) and Diamine B (127 mg, 1.44 mmol) in AcOH and THF (3:1.25 ml) at 0° C. was also prepared. The first mixture was added dropwise to the second mixture over 10 min at 0° C. Then the reaction mixture (which was yellowish in color) was allowed to warm to room temperature when it turned brown. After additional 24 h at room temperature, the final reaction mixture (around 10 ml) was directly transferred into a 1000-Dalton cut-off Spectra/Por®6 dialysis membrane (Repligen, USA) and dialyzed against 5 L acidified water (pH, 3-4); the acidified water was prepared by adding 3 ml of 1 M HCl to 5 L of Millipore water and the acidified water was replaced 3 times over a 24 h period. The polymer solution in the dialysis bag was transferred to a round bottomed flask and water was evaporated with a rotary evaporator (70° C., 1 h, 120 rpm) to yield a residue of PIM1D. To transfer the PIM1D for freeze-drying, 5 ml water was added to polymer solution and the concentrated PIM1D solution was decanted into a small falcon tube (15 ml) and then freeze-dried at −80° C. The PIM1D was characterized by water phase GPC and NMR (recorded on a Bruker Avance DPX-300 spectrometer at 300 MHz for 1H NMR and 75 MHz for 13C NMR) using deuterium dimethyl sulfoxide (DMSO-d₆).

### MTT Assay

Human mammary cell line MCF-7 cells were adjusted to desired density in Dulbecco's modified Eagle medium (DMEM) supplemented with 10% foetal bovine serum (FBS). Cells were seeded at 2×10\hat{ }4 cells/well in a 96-well plate and incubated without agitation at 37° C. for 24 hours. Subsequently, the supernatant was aspirated, compounds at desired concentration were added to each well and incubated for another 24 hours. The compound solution was then discarded, and cells were washed with PBS to remove any residual compound. MTT dye ((3-(4,5-dimethylthiazol-2-yl)-2,5-diphenyltetra zolium bromide)) was dissolved in pure DMEM medium at 1 mg/mL and added to the cells. The cells were further incubated for 3 hours at 37° C. Then the supernatant was aspirated, and 100 μL DMSO were added to solubilize the strongly pigmented formazan product. The absorbance at OD=570 nm were recorded using a plate reader and cell viability was calculated.

### BS EN 1656

S. aureus ATCC 6538, S. uberis ATCC 19436 and E. coli ATCC 10536 were used for the tests, as recommended by BS EN 1656 standard. Bacteria of 2^{nd} or 3^{rd} subculture were streaked out from TSA plates and inoculated into Tryptone NaCl diluent solution (0.1% tryptone and 0.85% NaCl) at 1.5 to 5×10\hat{ }8 CFU/mL. Compounds were dissolve in hard water (0.119 g MgCl₂, 0.277 g CaCl₂, 0.28 g NaHCO₃ in 1 L water) at desired concentration. 20 μL skimmed milk (100 g/L) were added into 96 well plate, followed by addition of 10 μL bacteria test suspension. The plate was mixed and incubated at 30° C. for 2 min. Subsequently, 80 μL product test solution were added and mixed well, incubated at 30° C. for 5 min, 10 min, 30 min, respectively. At desired time points, 20 μL of the product/milk/bacteria mixture were transferred to a new 96-well plate containing 160 μL neutralizer (Lecithin 3%, Tween 80 10% (w/v), Sodium Thiosulphate 0.3%) and 20 μL mQ water, mixed well and incubated at room temperature for 5 min to fully neutralize the compound. The mixture was then 10-fold serial dilute in Tryptone NaCl diluent solution, and plate onto TSA plates. Plates were enumerated after 24 hours incubation at 37° C.

### Example 2

### Farm Trial

The farm trial was performed following the recommended protocols for evaluating efficacy of postmilking teat germicides by National Mastitis Council of the United States [Nickerson, S. et al., NMC Annual Meeting Proceedings, 379-399 (2004)].

### Farm Condition

The trial was conducted in a dairy farm located at Jilin Province, Changchun city, Jiutai district, Longjia town, Xiaochengzi village, China. The trial was done in the local wintertime with outdoor temperature ranging from −10 to 10° C., from mid-March to early April. The farm houses over 500 dairy cows and is equipped with an automated milking system. Cows are housed in the barn and are milked at a separate milking facility with ambient temperature of 5 to 10° C. Milking is routinely done once per day at 2 pm by a skilled worker. Post-milking teat dipping with Iodine-based commercial products is practiced daily in the farm, and is ceased 10-days prior to the start of the trial on the experimental cows to avoid carryover effect and disturbance on the farm trial results.

### Setup

A 10-day acclimation period was implemented prior to the start of the experiment. Safety test starts on Day 11. First sampling was done before teat dip application (t=0 day). Teat dip was applied daily post-milking and continued for 5 days. 2^{nd} sampling was done on Day 16 (t=5 day). Samples collected: milk sample for quality check and residual check; teat surface sample for residual check.

Challenge test starts on Day 16. First sampling was done before milking to establish the baseline (t=0 day). Bacteria (5×10\hat{ }7 CFU/mL in TSB) was applied to teat right after each milking. Then teat dip was applied immediately. 2^{nd} and 3^{rd} sampling were done on Day 19 (t=3 day) and Day 21 (t=5 day), respectively. Samples collected: milk sample for quality check and CFU check.

### Preparation of Product Solution

The product stock solution was stored at 4° C. The product working solution was prepared fresh on the day of the experiment by diluting the stock solution (1/30 ratio) in sterile DI water to achieve a final concentration of 0.05% w/v active compound and 10% w/v glycerol.

### Preparation of Challenge Cultures for Efficacy Study

S. aureus ATCC 49525 was used in farm trial due to its close relevance to clinical bovine mastitis [Wall, R. J. et al., Nature Biotechnology, 23(4):445-451 (2005)]. A single colony was streaked out from agar plate into Trypticase Soy Broth (TSB) and incubated overnight under shaking at 37° C. The overnight culture was subcultured 1:100 into fresh TSB cultures and incubated for 3 hours to obtain exponentially growing bacteria. Bacterial cells were pelleted by centrifugation (3,000-4,000 g for 15 min), washed twice with 0.1% proteose-peptone and diluted to ˜5×10⁷ CFU/ml in fresh TSB. The challenge suspension containing 5×10⁷ CFU/ml in TSB was prepared immediately before use when practical.

### Premilking Udder Preparation

Premilking udder preparation consists of the use of single service water-moistened towels (free of sanitizer, one towel per teat) to wet and clean the teats prior to fore-stripping. Fore-stripping was accomplished by expressing three squirts of milk.

### Experimental Exposure of Teats and Teat Disinfection

### (1) Safety Study

Teat dip was applied to the distal 25 mm of teats immediately after milking. Each teat was dipped once with the product in a conventional foam dip cup.

### (2) Efficacy Study

Teats were challenged by the testing organism suspension immediately after milking. Each teat was challenged by immersion to a depth of approximately 25 mm in a conventional foam dip cup containing freshly prepared TSB suspension of the test organism S. aureus. Challenge was performed once daily for 5 consecutive days. Teat dip was applied to the distal 25 mm of teats immediately after exposure to the challenge suspension.

### Sampling Procedures

### (1) Safety Study

### Polymer Absorption on Skin

To determine the polymer absorption on teat skin, swab sample was taken daily starting from the onsite of the polymer product. After cleaning the teats, the samples were taken using the wet and dry swab technique in accordance with DIN 10113-1: 1997-07. A cotton wool swab moistened with sterile 0.25% Ringer's solution is moved around the teat at a distance of 1 cm from the teat canal orifice. After that the same procedure was performed with a dry cotton wool swab (ultrafine, dry swab). Both swabs were shortened and inserted into one test tube containing 2 mL of the sterile 0.25% Ringer's solution.

The solution was further transferred to lab to determine residual antimicrobials using Delvo test Kit. Briefly, 200 μl of the testing samples were added to the Delvo test ampoules and are incubated at 63° C. for 3 hours. The color change of the solid agar at the bottom of Delvo test ampoules was recorded.

### Milk Sampling

All milk samples were collected immediately prior to a regular automated milking. Briefly, three or four streams of foremilk were discarded from each quarter before sanitizing teat ends with cotton swabs and collecting samples. Approximately 10 mL of milk samples from each teat were collected daily starting from the onsite of the polymer product. To determine the polymer residual in milk, the milk samples were transferred to lab to determine residual antimicrobials using Delvo test Kit. To determine the milk quality (e.g., somatic cell count), milk samples were passed and tested by qualified testing labs within 24 hours.

### (2) Efficacy Study

The sampling procedures and testing protocols follows that of the Safety study as previously mentioned. The teat surface samples and milk samples were examined microbiologically according to standard procedures. The number of microbes was numerated and recorded accordingly.

### Criteria for Diagnosing Infections

A new IMI in a quarter is diagnosed when the same bacterial species is isolated from 1) two consecutive samples during the trial (>500 CFU/ml); 2) a single sample from a quarter with clinical mastitis (>100 CFU/ml); or 3) three consecutive samples during the trial (>100 CFU/ml).

### Example 3

### Application as Post-Milking Teat Dip to Prevent Bovine Mastitis

We first tested the minimal inhibitory concentrations (MICs) of PIM1 against mastitis-causing pathogens, including Gram-positive bacteria S. aureus, S. uberis and Gram-negative bacteria E. coli. PIM1 showed broad-spectrum antibacterial activity with low MICs (Table 2).

TABLE 2
MIC against mastitis-causing pathogens

MIC (μg/mL)

| | S. aureus | S. uberis | E. coli |
|---|---|---|---|
| Compound | ATCC 6538 | ATCC 19436 | ATCC 10536 |
| PIM1 | 1 | 1 | 4 |

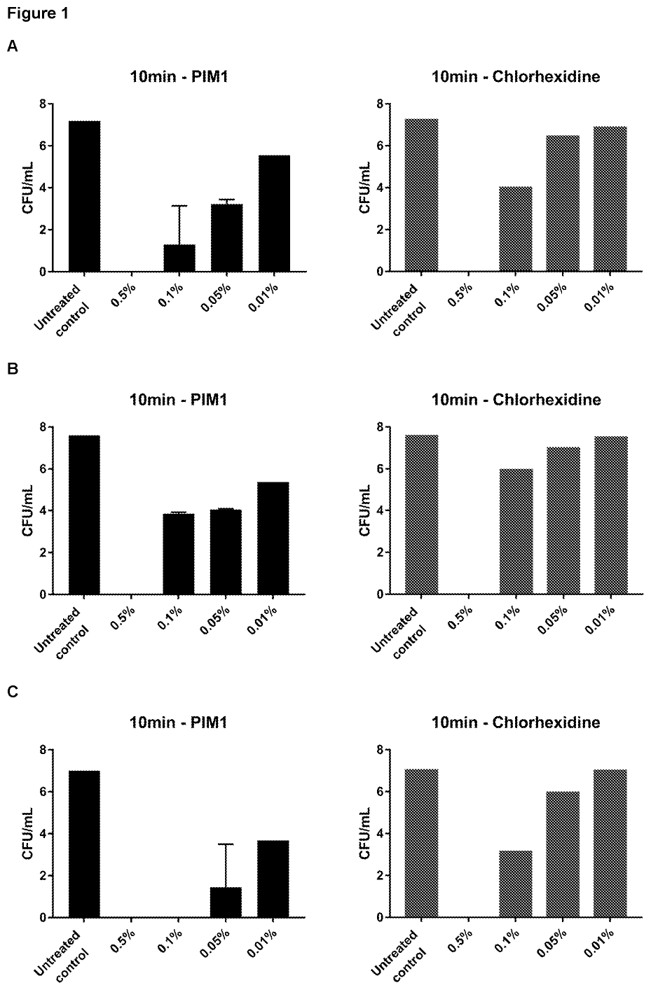

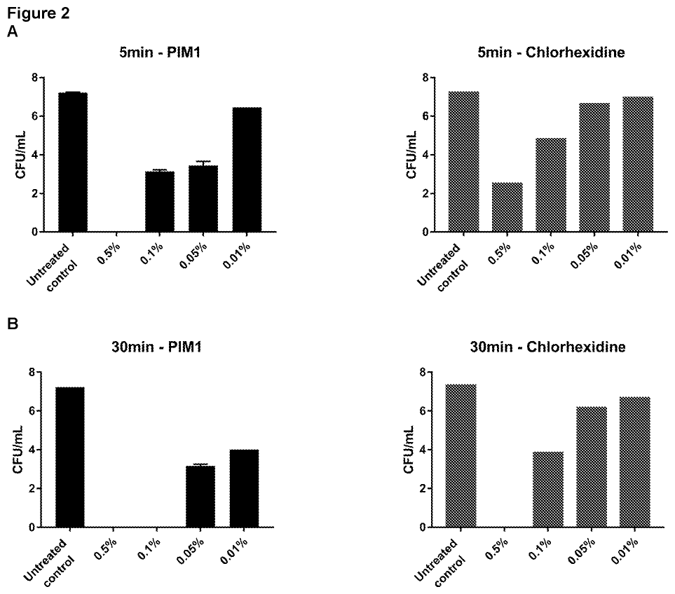

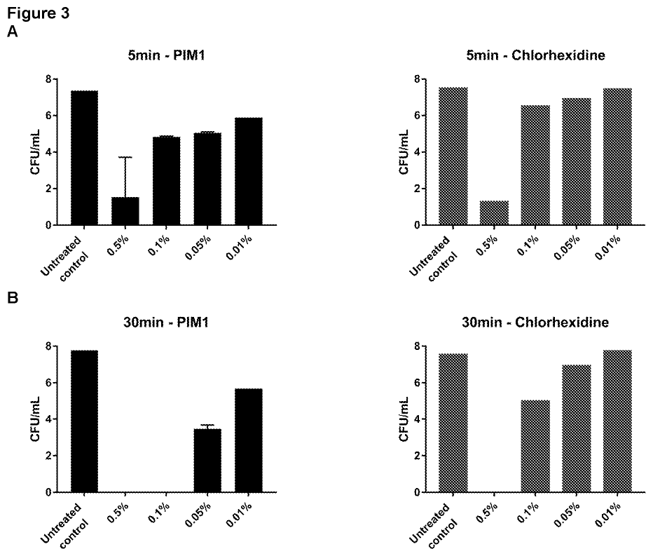

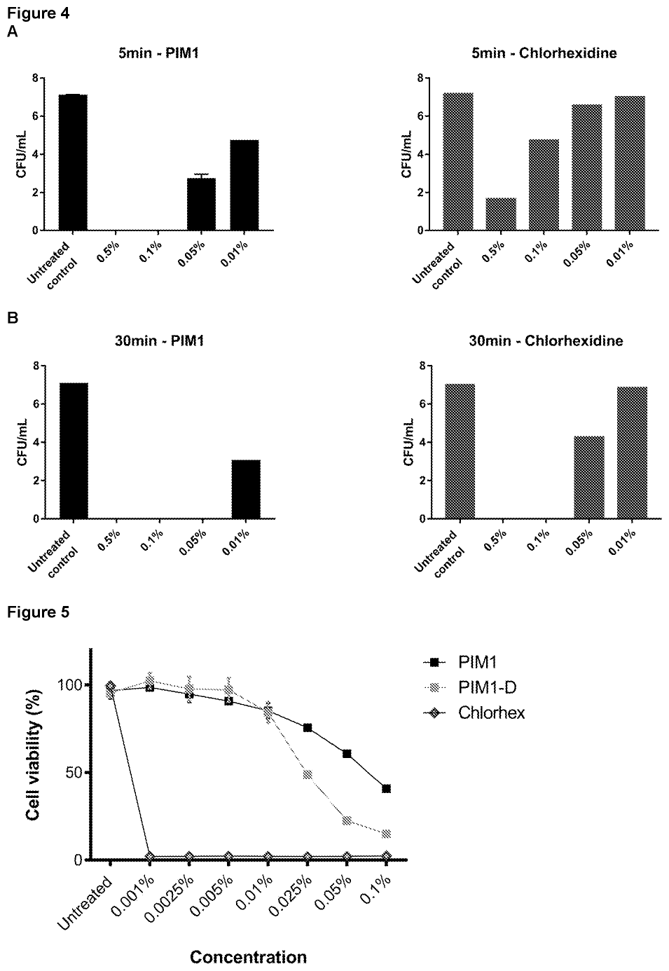

We then tested the bactericidal activity of PIM1 and a commercially used antimicrobial chlorhexidine against these pathogens following industrial standard method BS EN 1656 with minor modifications. In the presence of 18% milk, PIM1 retained excellent bactericidal activity within 10 minutes, achieving >5 log₁₀ reduction at 0.1%, 0.5%, and 0.05% w/v against S. aureus, S. uberis and E. coli, respectively (FIGS. 1A, 1B and 1C, respectively). This is superior to that of chlorhexidine, which only achieves the abovementioned log reduction at 0.5%. Moreover, we also conducted the test for 5 minutes and 30 minutes, and confirmed that PIM1 showed superior bactericidal activity than chlorhexidine at all the time points against S. aureus (FIGS. 2A and 2B), S. uberis (FIGS. 3A and 3B) and E. coli (FIGS. 4A and 4B). To demonstrate the biocompatibility towards human mammary MCF-7 cells, we conducted MTT assays and confirmed that, over a concentration range of 0.001%-0.1% w/v, PIM1 (solid black box) and PIM1D (solid grey box) showed much reduced toxicity compared to chlorhexidine (diamond) (FIG. 5).

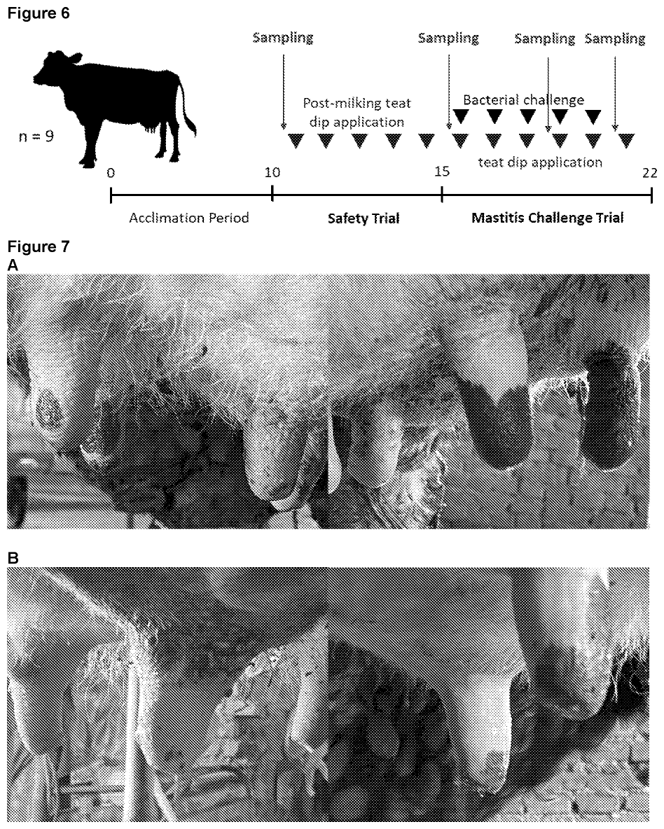

To further prove the safety and efficacy of PIM1 and PIM1D, we conducted a farm trial following the recommended protocols for evaluating efficacy of post-milking teat germicides by national mastitis council of the United States. The farm trial is divided into two parts, to confirm: (1) Safety of the PIM1 and PIM1D based teat dips and (2) Efficacy in preventing new Intramammary infection (IMI) in dairy cows following experimental exposure of teats to mastitis pathogens. A schematic of the farm trail is illustrated in FIG. 6. The methods used are described in Example 2.

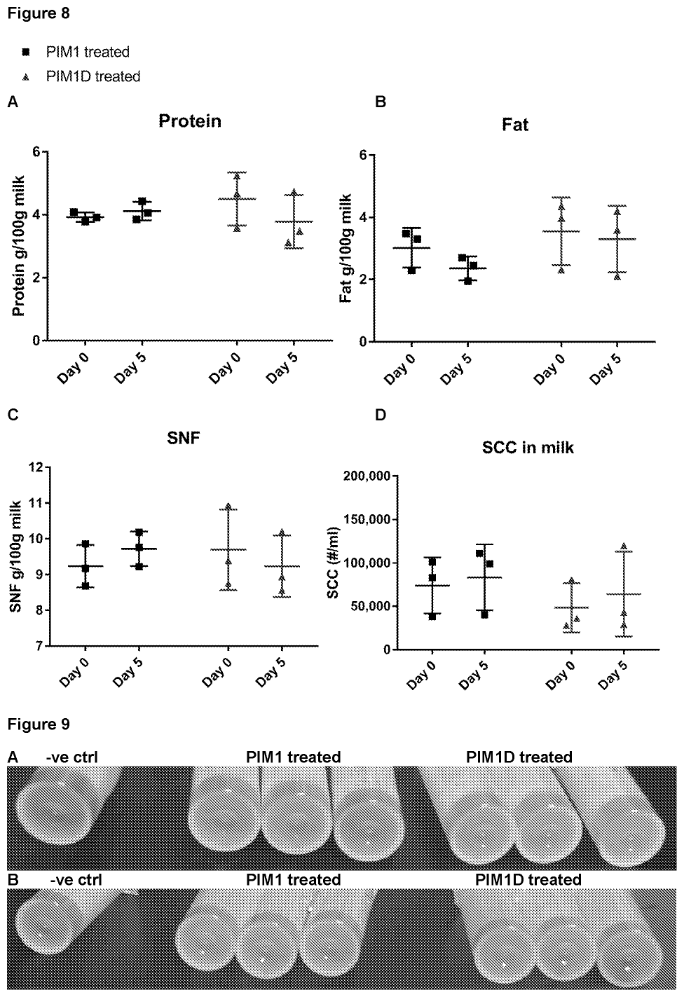

In the safety test, upon the 5-day continuous application, no irritation response (e.g., redness, edema, roughness, additional lesion) is observed for the PIM1- and PIM1D-treated teats (FIGS. 7A and 7B, respectively). The cows also did not show abnormal behaviours (e.g., restless, kicking or rubbing at abdomen area), which are signs of irritating and itching teat skin. Both PIM1 and PIM1D-based teat dips did not result in changes in milk composition, including protein, fat, solid non-fat (SNF) and Somatic cell counts (SCCs) contents (FIGS. 8A, 8B, 8C and 8D, respectively). All the changes are not statistically significant (p>0.05). Moreover, no polymer residuals are detected in the teat surface (FIG. 9A) and milk samples (FIG. 9B) as confirmed by Delvo Test [Stead, S. et al., International Dairy Journal 18(1):3-11 (2008)](<10 ppm, limit of detection), indicating that both compounds can be easily washed off during the premilking cleaning steps to avoid carryover of compounds into milk.

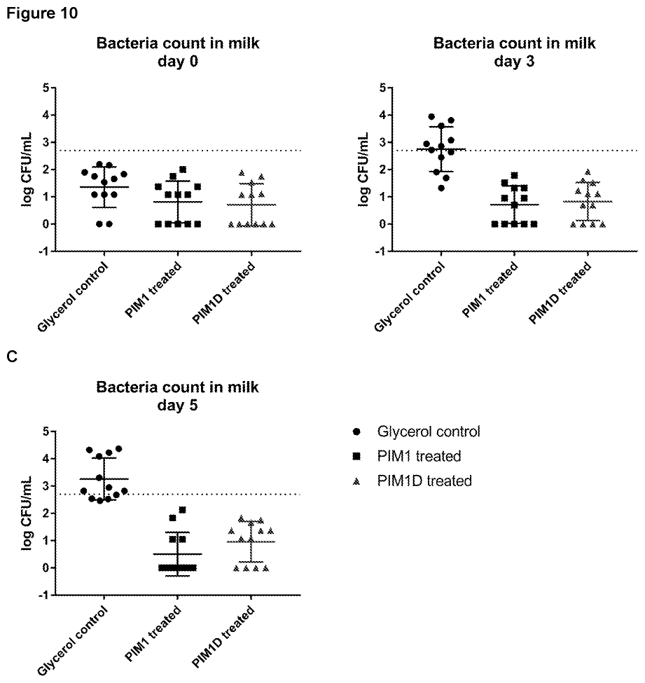

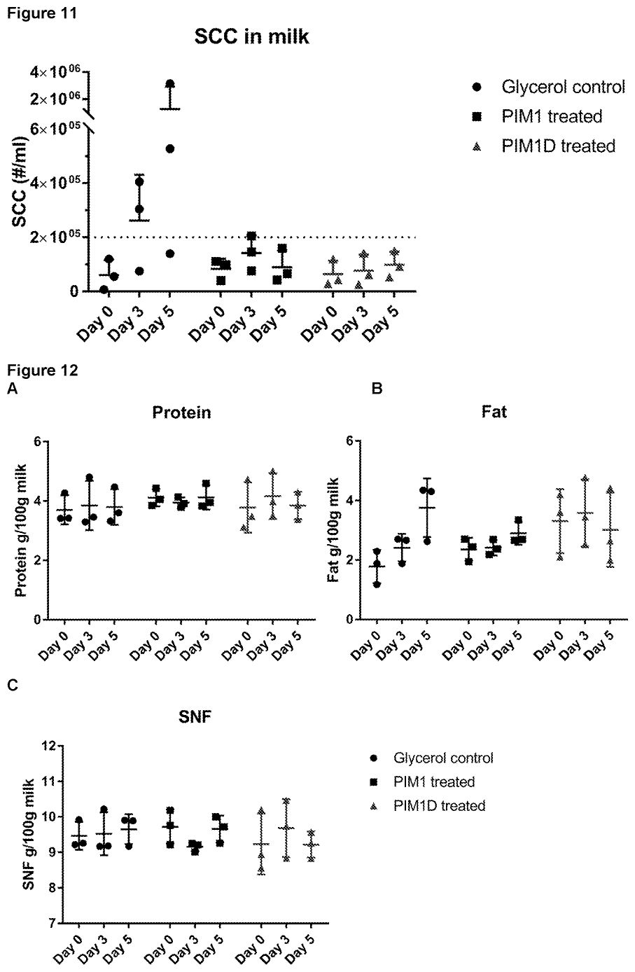

In the efficacy test, PIM1 and PIM1D based teat dips successfully prevented mastitis upon repeated S. aureus exposure for 0, 3 and 5 days, whereas cows from glycerol control group developed mastitis overtime. Based on the guideline of National Mastitis Council of the United States [Nickerson, S. et al., NMC Annual Meeting Proceedings, 379-399 (2004)], the criteria for mastitis is defined as >500 CFU/mL in milk samples (indicated by the dotted line in FIG. 10). The onset of mastitis in glycerol control groups are also evidenced by the SCC numbers in milk samples upon bacteria challenge (FIG. 11). Based on the criteria for mastitis (SCC>200,000/ml, dotted line), cows in glycerol control group developed mastitis overtime. PIM1 and PIM1D-based teat dip kept SCC counts low in milk, confirming that both compounds prevented mastitis from exposure to contagious bacteria. The milk quality of each cow is also monitored by examining the milk composition (protein, fat, SNF). As shown in FIG. 12, milk from PIM1 and PIM1D treated cows have no statistically significant changes in milk composition. Though changes of protein and SNF content remain insignificant for the glycerol control group, the fat content increases significantly indicating the possible onset of mastitis [Petroski, K., Milk composition changes during mastitis (2006)](p<0.01).

### SUMMARY

The present invention provides the use of PIM compounds as post-milking teat dip in methods to prevent bovine mastitis. PIM1 and PIM1D compounds demonstrated superior bactericidal activity in the presence of milk and biocompatibility towards mammary cells in comparison to commercial cationic antimicrobial chlorhexidine. These post-milking teat dip products also showed in vivo safety and efficacy in a farm trial.

### REFERENCES

Institution, B. S., Chemical Disinfectants and Antiseptics: Quantitative Suspension Test for the Evaluation of Bactericidal Activity of Chemical Disinfectants and Antiseptics Used in Food, Industrial, Domestic and Institutional Areas: Test Method and Requirements (phase 2, Step 1). British Standards Institution: 2019.
Nickerson, S.; Saxon, A.; Fox, L.; Hemling, T.; Hogan, J.; Morelli, J.; Oliver, S.; Owens, W.; Pawlak, M.; Petersson, L. In National Mastitis Council: Recommended protocols for evaluating efficacy of postmilking teat germicides, NMC Annual Meeting Proceedings, Citeseer: 2004; pp 379-399.
Petrovski, K., Milk composition changes during mastitis. 2006.
Stead, S.; Ashwin, H.; Richmond, S.; Sharman, M.; Langeveld, P.; Barendse, J.; Stark, J.; Keely, B., Evaluation and validation according to international standards of the Delvotest® SP-NT screening assay for antimicrobial drugs in milk. International dairy journal 2008, 18 (1), 3-11.
Wall, R. J.; Powell, A. M.; Paape, M. J.; Kerr, D. E.; Bannerman, D. D.; Pursel, V. G.; Wells, K. D.; Talbot, N.; Hawk, H. W., Genetically enhanced cows resist intramammary Staphylococcus aureus infection. Nature Biotechnology 2005, 23 (4), 445-451.
Zhong, W.; Shi, Z.; Mahadevegowda, S. H.; Liu, B.; Zhang, K.; Koh, C. H.; Ruan, L.; Chen, Y.; Zeden, M. S.; Pee, C. J. E.; Marimuthu, K.; De, P. P.; Ng, O. T.; Zhu, Y.; Chi, Y. R.; Hammond, P. T.; Yang, L.; Gan, Y.-H.; Pethe, K.; Greenberg, E. P.; Grundling, A.; Chan-Park, M. B., Designer broad-spectrum polyimidazolium antibiotics. Proceedings of the National Academy of Sciences 2020, 117 (49), 31376-31385.

1. A method of prophylaxis or treatment of mastitis in a mammal, comprising administering an efficacious amount of a composition comprising one or more polyimidazolium (PIM) compounds to said mammal, wherein the one or more polyimidazolium (PIM) compounds is selected from the group consisting of:

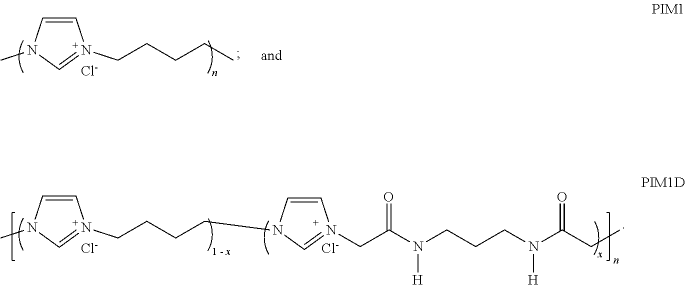

2. The method of claim 1, wherein:
a) for PIM1 n is a number selected from 2 to 10 and the number average molecular weight is from 1,000 to 2,000 Daltons;
b) for PIM1D n is a number selected from 2 to 10, x is about 25%, and the number average molecular weight is from 1,000 to 2,000 Daltons.

3. The method of claim 2, wherein:
a) for PIM1 n=9 and the number average molecular weight is 1562 Daltons; and
b) for PIM1D n=10, x is about 25% and the number average molecular weight is 1856 Daltons.

4. The method of claim 1, wherein the polyimidazolium compound is a salt, such as a chloride salt.

5. The method of claim 1, wherein the composition comprises at least 0.05% w/v polyimidazolium compound.

6. The method of claim 1, wherein the composition further comprises glycerol.

7. The method of claim 6, wherein the composition comprises glycerol in an amount of 10% w/v or less.

8. The method of claim 1, wherein the mammal is selected from the group comprising cows, pigs, goats, sheep and humans.

9. The method of claim 8, wherein the mammal is a cow.

10. The method of claim 1, comprising:
applying the composition to one or more teats of said mammal.

11. The method of claim 10, wherein the composition is applied daily post-milking.

12. A composition comprising one or more polyimidazolium (PIM) compounds formulated to prevent or treat mastitis in a mammal, wherein said one or more polyimidazolium (PIM) compounds are selected from the group consisting of:

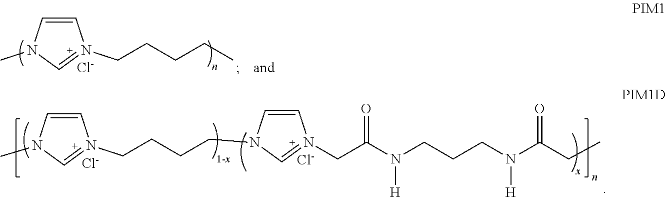

13. The composition of claim 12, wherein:
a) for PIM1 n is a number selected from 2 to 10 and the number average molecular weight is from 1,000 to 2,000 Daltons;
b) for PIM1D n is a number selected from 2 to 10, x is about 25%, and the number average molecular weight is from 1,000 to 2,000 Daltons.

14. The composition of claim 13, wherein:
a) for PIM1 n=9 and the number average molecular weight is 1562 Daltons; and
b) for PIM1D n=10, x is about 25% and the number average molecular weight is 1856 Daltons.

15. The composition of claim 12, wherein the polyimidazolium compound is a salt, such as a chloride salt.

16. The composition of claim 12, wherein the composition comprises at least 0.05% w/v polyimidazolium compound.

17. The composition of claim 12, wherein the composition further comprises glycerol.

18. The composition of claim 17, wherein the composition comprises glycerol in an amount of 10% w/v or less.

19. The composition of claim 12, wherein the mammal is selected from the group comprising cows, pigs, goats, sheep and humans.

20. The composition of claim 19, wherein the mammal is a cow.

21.-23. (canceled)
